(19) United States
(12) Patent Application Publication
(10) Pub. No.: US 2024/0199948 A1
(43) Pub. Date: Jun. 20, 2024

(54) POLYMER

(71) Applicant: Sumitomo Chemical Co., Ltd., Tokyo (JP)

(72) Inventors: Philip Stackhouse, Godmanchester (GB); Kiran Kamtekar, Cambridgeshire (GB); Nazrul Islam, Cambridgeshire (GB)

(73) Assignee: Sumitomo Chemical Co., Ltd., Tokyo (JP)

(21) Appl. No.: 18/284,849

(22) PCT Filed: Mar. 30, 2022
(86) PCT No.: PCT/EP2022/058397

§ 371 Date: Sep. 28, 2023

(30) Foreign Application Priority Data
Mar. 30, 2021 (GB) 2104548.9

(51) Int. Cl. C09K 11/06; C08G 61/12; C12Q 1/48; C12Q 1/6806; C12Q 1/6825; C12Q 1/6869

(52) U.S. Cl. CPC C09K 11/06; C08G 61/122; C12Q 1/485; C12Q 1/6806; C12Q 1/6825; C12Q 1/6869; C08G 2261/148; C08G 2261/334; C08G 2261/52; C08G 2261/94; C09K 2211/1483; G01N 2333/9126

(57) ABSTRACT

A conjugated polymer comprising a first repeat unit substituted with at least three ionic groups.

Light-emitting polymers for marking a target analyte are known. For example, WO 2018/060722 discloses a particle comprising silica and a light-emitting polymer comprising a backbone and polar groups pendant from the backbone.

WO2018/11177 discloses water-soluble polymeric dyes and polymeric tandem dyes including branched non-ionic water soluble groups.

Qin et al, “High solubility and photoluminescence quantum yield water-soluble polyfluorenes with dendronized amino acid side chains: synthesis, photophysical, and metal ion sensing properties” J. Mater. Chem., 2010, 20, 7957-7964 discloses anionic water-soluble dendronized conjugated polymers.

Yu et al, “Water-Soluble Dendritic-Conjugated Polyfluorenes: Synthesis, Characterization, and Interactions with DNA” Journal of Polymer Science Part A (Polymer Chemistry), Volume 46, Issue 22 15 Nov. 2008 pages 7462-7472 discloses cationic dendritic conjugated polyfluorenes.

### SUMMARY

In some embodiments, the present disclosure provides a conjugated polymer comprising a first repeat unit substituted with at least three ionic groups.

Optionally, each ionic group is an anionic group.

Optionally, the polymer is a copolymer comprising the first repeat unit and at least one further repeat unit.

Optionally, the first repeat unit is substituted with at least one ionic substituent carrying at least 2 ionic groups.

Optionally, the at least one ionic substituent is substituted with at least three ionic groups.

Optionally, the first repeat unit substituted with at least two of the ionic substituents.

Optionally, the ionic substituent has formula (I):

-L-(R¹)x (I)

wherein each R¹ is independently an ionic group: x is at least 1; and L is a linking group linking the ionic group or ionic groups to R¹ to the first repeat unit.

Optionally. the first repeat unit is selected from repeat units of formulae (V)-(XVI):

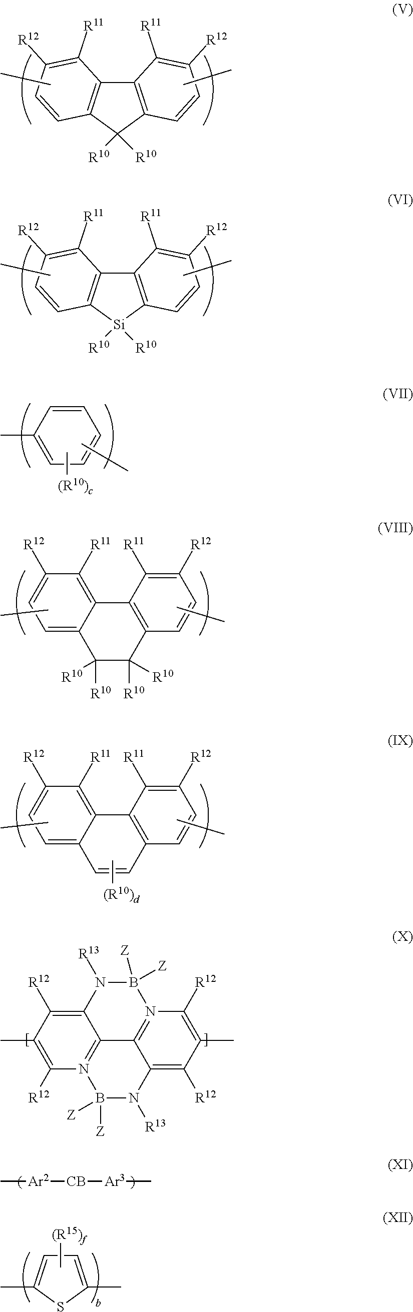

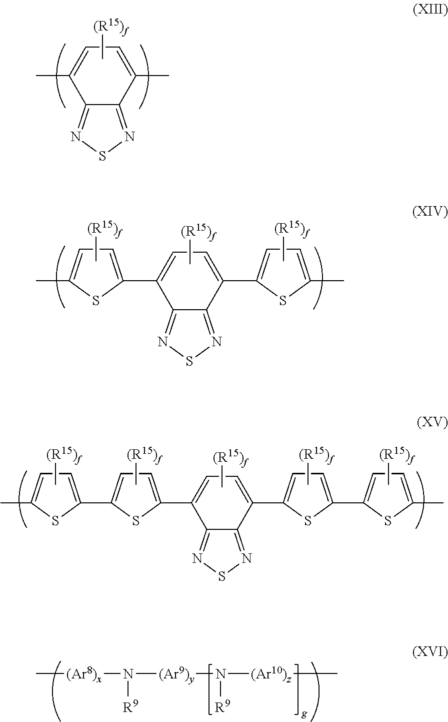

wherein:
R¹⁰ in each occurrence is independently a substituent;
R¹¹ in each occurrence is independently H or a substituent and two R¹¹ groups may be linked to form a ring;
R¹² independently in each occurrence is H or a substituent;
R¹³ independently in each occurrence is a C₁₋₂₀ hydrocarbyl group;
R¹⁵ independently in each occurrence is a substituent;
Z in each occurrence is independently a substituent.
c is 0, 1, 2, 3 or 4;
d is 0, 1 or 2;
f independently in each occurrence is 0, 1 or 2;
Ar⁸, Ar⁹ and Ar¹⁰ in each occurrence are independently selected from substituted or unsubstituted arylene or heteroarylene;
g is 0, 1 or 2;
R⁹ independently in each occurrence is a substituent,
x, y and z are each independently 1, 2 or 3;
Ar² and Ar³ each independently represent a C₆₋₂₀ arylene group or a 5-20 membered heteroarylene group which is unsubstituted or substituted with one or more substituents; and
CB represents a conjugation-breaking group which does not provide a conjugation path between Ar² and Ar³.

In some embodiments, the present disclosure provides a light-emitting particle comprising the conjugated polymer according to any one of the preceding claims

Optionally, the particle comprises a matrix material.

Optionally, the matrix material is silica. Optionally, formation of the light-emitting particle comprises reacting a material for forming the silica in the presence of the conjugated polymer dissolved in an alcoholic solvent.

In some embodiments, the present disclosure provides a light-emitting marker comprising the conjugated polymer or the light-emitting particle according to any one of the preceding claims and a binding group comprising a biomolecule.

In some embodiments, the present disclosure provides a precursor of a light-emitting marker as described herein comprising a functional group capable of binding to the biomolecule.

Optionally, the functional group comprises biotin.

In some embodiments, the present disclosure provides a method of forming a light-emitting marker as described herein comprising binding the biomolecule to the functional group of the precursor as described herein.

In some embodiments, the present disclosure provides a formulation comprising the conjugated polymer, light-emitting particle or precursor as described herein dissolved or dispersed in one or more solvents.

In some embodiments, the present disclosure provides a method of identifying a target analyte in a sample, the method comprising irradiating the sample to which has been added a light-emitting marker as described herein configured to bind to the target analyte; and detecting emission from the light-emitting marker,

Optionally, the method is a flow cytometry method and the target analyte is a target cell.

In some embodiments, the present disclosure provides a method of sequencing nucleic acids comprising contacting a primed template nucleic acid molecule with a polymerase and a test nucleotide;
incorporating the test nucleotide into a primed strand of the primed template only if it comprises a base complementary to the next base of the template strand;
irradiating the primed strand; and
determining from luminance of the primed strand if the test nucleotide has been incorporated into the primed strand,
wherein the test nucleotide of the irradiated primed strand is bound to a conjugated polymer, light-emitting particle or light-emitting marker as described herein configured to bind to the test nucleotide.

### DETAILED DESCRIPTION

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” Additionally, the words “herein,” “above,” “below,” and words of similar import, when used in this application, refer to this application as a whole and not to any particular portions of this application. Where the context permits, words in the Detailed Description using the singular or plural number may also include the plural or singular number respectively.

The word “or,” in reference to a list of two or more items, covers all of the following interpretations of the word: any of the items in the list, all of the items in the list, and any combination of the items in the list. References to a specific atom include any isotope of that atom unless specifically stated otherwise.

The teachings of the technology provided herein can be applied to other systems, not necessarily the system described below. The elements and acts of the various examples described below can be combined to provide further implementations of the technology. Some alternative implementations of the technology may include not only additional elements to those implementations noted below, but also may include fewer elements.

These and other changes can be made to the technology in light of the following detailed description. While the description describes certain examples of the technology, and describes the best mode contemplated, no matter how detailed the description appears, the technology can be practiced in many ways. As noted above, particular terminology used when describing certain features or aspects of the technology should not be taken to imply that the terminology is being redefined herein to be restricted to any specific characteristics, features, or aspects of the technology with which that terminology is associated. In general, the terms used in the following claims should not be construed to limit the technology to the specific examples disclosed in the specification, unless the Detailed Description section explicitly defines such terms. Accordingly, the actual scope of the technology encompasses not only the disclosed examples, but also all equivalent ways of practicing or implementing the technology under the claims.

To reduce the number of claims, certain aspects of the technology are presented below in certain claim forms, but the applicant contemplates the various aspects of the technology in any number of claim forms.

In the following description, for the purposes of explanation, numerous specific details are set forth in order to provide a thorough understanding of implementations of the disclosed technology. It will be apparent, however, to one skilled in the art that embodiments of the disclosed technology may be practiced without some of these specific details.

The present inventors have found that high water and/or methanol solubility of a conjugated polymer may be achieved by substituting a first repeat unit of the polymer with at least three ionic groups.

Solubility of a conjugated polymer as described herein is preferably greater than 1 mg/ml, optionally at least 3 mg/ml or at least 5 mg/ml in at least one of methanol and water, preferably in methanol.

To determine solubility of a polymer as described herein, the solid polymer is weighed out into a glass vial. The required amount of solvent is added followed by a small magnetic stirrer. Then the vial is tightly capped and put on a preheated hot plate at 60° C. with stirring for 30 minutes. The polymer solution is allowed to cool to room temperature before use. The polymer solution can also be prepared by sonicating the polymer containing vial for 30 min at room temperature. The solubility of polymer was tested by visual observation and under white and 365 nm UV light.

The polystyrene-equivalent weight-average molecular weight (Mw) of the light-emitting polymers described herein may be 1×10³ to 1×10⁸, and preferably 1×10⁴ to 1×10⁷. 3 or more ionic substituents of the first repeat unit may enhance the solubility of the polymer in water and/or methanol as compared to a repeat unit with fewer ionic substituents.

### Ionic Groups

The conjugated polymer includes a first repeat unit substituted with at least three ionic groups R¹.

In some embodiments, each ionic group is directly bound to the first repeat unit.

The ionic group R¹ may be anionic or cationic, preferably anionic.

The ionic group R¹ is preferably monovalent.

Exemplary anionic groups are —COO⁻, a sulfonate group; hydroxide; sulfate; phosphate; phosphinate; or phosphonate.

An exemplary cationic group is —N(R⁶)₃⁺ wherein R⁶ in each occurrence is H or C₁₋₁₂ hydrocarbyl. Preferably, each R⁶ is a C₁₋₁₂ hydrocarbyl.

A C₁₋₁₂ or C₁₋₂₀ hydrocarbyl group as described anywhere herein is optionally selected from a linear, branched or cyclic alkyl, optionally C₁₋₂₀ alkyl; unsubstituted phenyl; and phenyl substituted with one or more C₁₋₁₂ alkyl groups.

The polymer comprises counterions, preferably monovalent counterions, to balance the charge of the ionic groups.

In the case of an anionic group R¹, the cation counterion M⁺ is optionally a metal cation, optionally Li⁺, Na⁺, K⁺, Cs⁺, preferably Cs⁺, or an organic cation, optionally ammonium, such as tetraalkylammonium, ethylmethyl imidazolium or pyridinium.

In the case of a cationic group R¹, the anion counterion is optionally a halide; a sulfonate group, optionally mesylate or tosylate; hydroxide; carboxylate; sulfate; phosphate; phosphinate; phosphonate; or borate.

In some embodiments, at least one ionic substituent carrying one or more ionic groups is bound to the first repeat unit. An ionic substituent may be a substituent of formula (I):

-L-(R¹)x (I)
wherein each R¹ is independently an ionic group; x is at least 1; and L is a linking group linking the ionic group or ionic groups to R¹ to the first repeat unit.

Preferably, x is at least 2, optionally 2, 3 or 4. If x is at least 2 then R¹ in each occurrence may be the same or different, preferably the same.

Preferably, R¹ is a monovalent ionic group.

In some embodiments, L comprises or consists of one or more C₆₋₂₀ aromatic groups, preferably one or more benzene rings. Other than ionic groups R¹, each aromatic group may be unsubstituted or substituted with one or more non-ionic substituents R².

Exemplary ionic substituents comprising one or more C₆₋₂₀ aromatic groups include, without limitation:

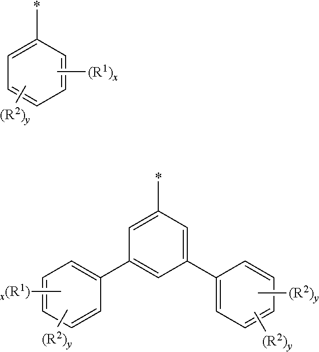

wherein R¹ and x are as described above; R² is a non-ionic group; y is 0 or a positive integer, optionally 1 or 2, preferably 0 or 1; and * is a point of attachment to the first repeat unit.

Exemplary groups of formula (Ia) and (Ib) are:

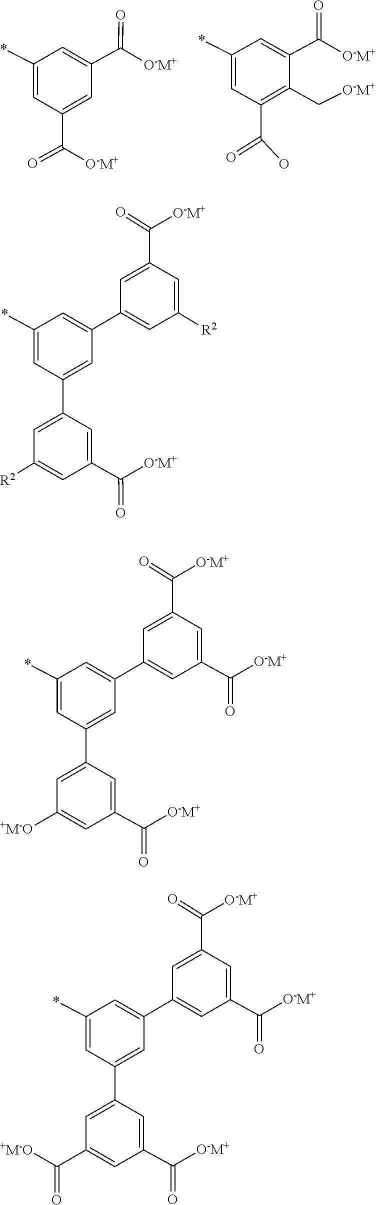

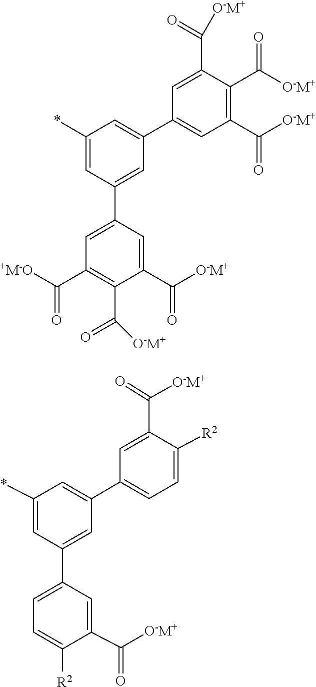

Optionally, each R² is independently selected from F; CN; NO₂; and C₁₋₃₀ alkyl wherein one or more non-adjacent, non-terminal C atoms of a C₂₋₃₀ alkyl may be replaced with O, S, COO or CO and one or more H atoms of the alkyl may be replaced with F. A preferred group of R² is a group comprising one or more ether units, more preferably a group of formula (II):

—(R⁵)ᵤ—O(R⁵O)ᵥ—R⁶ (II)

wherein R⁵ in each occurrence is a C₁₋₁₀ alkylene group, optionally a C₁₋₅ alkylene group, more preferably ethylene; R⁶ is H or C₁₋₅ alkyl; u is 0 or 1; and v is 0 or a positive integer, preferably 1-10. Preferably, v is at least 2.

By “non-terminal C atom” of an alkyl group as used herein means a C atom other than the methyl group at the end of an n-alkyl chain or the methyl groups at the ends of a branched alkyl chain.

In some embodiments, the ionic substituent comprises or consists of a dendron carrying a plurality of ionic groups. The dendron may comprise a core group and n dendrite generations wherein each of the nth generation dendrites are each substituted with at least one ionic group. n is optionally 1-5.

A first generation dendrimer (n=1) may have formula (III):

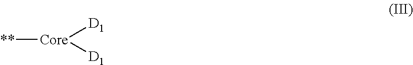

wherein each D1 is a first generation dendrite comprising at least one ionic group; and ** is a point of attachment to the first repeat unit or a substituent of the first repeat unit, e.g. a phenyl substituent of the first repeat unit.

A second generation dendrimer (n=2) may have formula (IV):

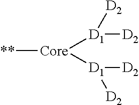

wherein each D1 is a first generation dendrite; and each D2 is a second generation dendrite comprising at least one ionic group.

If n>2 then it will be understood that an nth generation dendrimer may be formed by reacting ionic groups of the n-1 generation dendrimer, for example a carboxylate group which is reacted with an amine to convert the n-1 dendrite from a carboxylate group to an amide.

In some embodiments, the first generation dendrimer has formula:

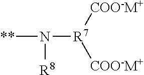

wherein R⁷ is a C₁₋₅ alkylene; R⁸ is H or a C₁₋₁₂ hydrocarbyl group, preferably H.

An n-generation dendrimer may be formed by reacting the n-1 generation dendrimer with a compound of formula:

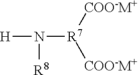

wherein R⁹ is a substituent, e.g. a C₁₋₁₂ hydrocarbyl group, followed by conversion of COOR⁹ to COO⁻M⁺. Exemplary dendrons include:

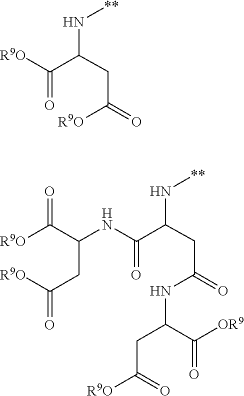

### Polymer Repeat Units

The polymer may consist only of one or more first repeat units. Optionally, the polymer is a homopolymer of a first repeat unit.

The polymer may be a copolymer comprising one or more first repeat units, preferably only one first repeat unit, and one or more further repeat units. Further repeat units may be unsubstituted or may be substituted with 0, 1 or 2 ionic groups, preferably no ionic groups, and/or one or more non-ionic substituents.

Preferably, at least 10 mol %, optionally at least 30 mol %, of the repeat units of a copolymer are first repeat units.

The first and further repeat units may be selected from, without limitation, repeat units of formulae (V)-(XVI):

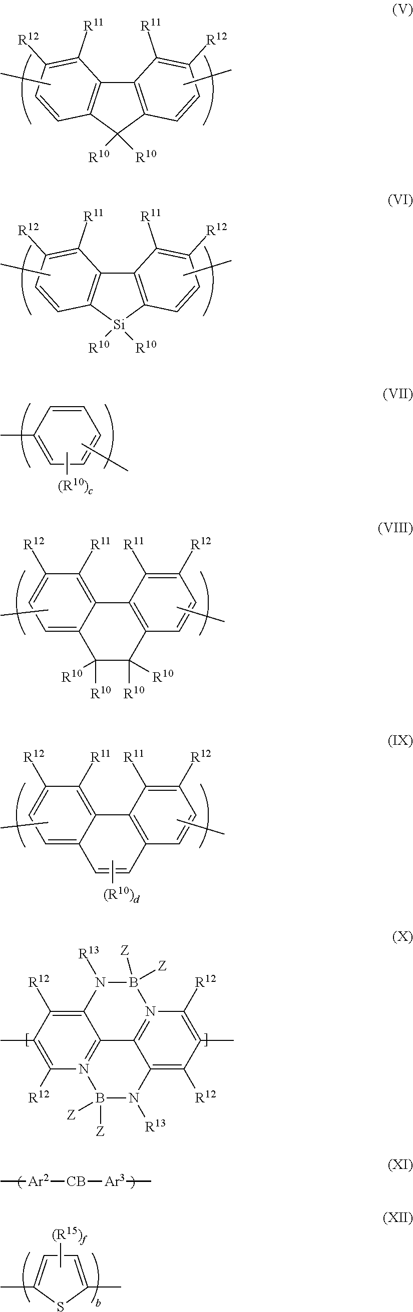

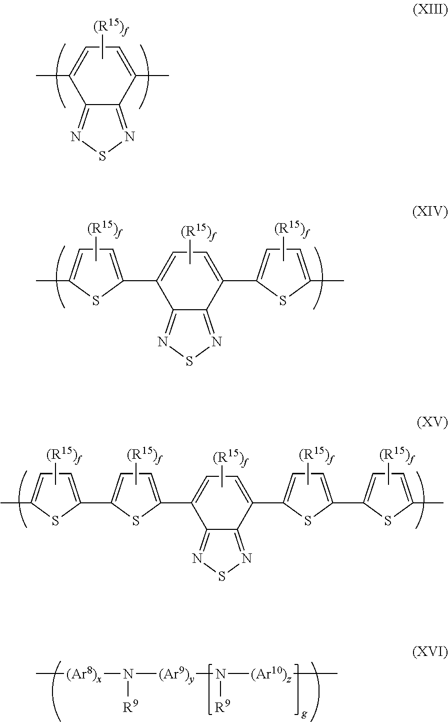

R¹⁰ in each occurrence is independently a substituent.

R¹¹ in each occurrence is independently H or a substituent and two R¹¹ groups may be linked to form a ring.

R¹² independently in each occurrence is H or a substituent.

R¹³ independently in each occurrence is a C₁₋₂₀ hydrocarbyl group.

R¹⁵ independently in each occurrence is a substituent.

Z in each occurrence is independently a substituent. Preferably, Z independently in each occurrence is selected from the group consisting of branched, linear or cyclic C₁₋₂₀ alkyl; phenyl which is unsubstituted or substituted with one or more substituents, e.g. one or more C₁₋₁₂ alkyl groups; and F.
c is 0, 1, 2, 3 or 4, preferably 1 or 2.
d is 0, 1 or 2.
f independently in each occurrence is 0, 1 or 2.

Ar⁸, Ar⁹ and Ar¹⁰ in each occurrence are independently selected from substituted or unsubstituted arylene or heteroarylene.

The value of g is 0, 1 or 2, preferably 0 or 1.

R⁹ independently in each occurrence is a substituent, and x, y and z are each independently 1, 2 or 3.

Ar² and Ar³ each independently represent a C₆₋₂₀ arylene group or a 5-20 membered heteroarylene group which is unsubstituted or substituted with one or more substituents;

CB represents a conjugation-breaking group which does not provide a conjugation path between Ar² and Ar³. CB does not provide any conjugation path between Ar² and Ar³. Optionally, CB does not provide a path of alternating single and double bonds between Ar¹ and Ar². Optionally, CB is a C₁₋₂₀ branched or linear alkylene group wherein one or more H atoms may be replaced with F and one or more non-adjacent C atoms of the alkylene group may be replaced with O, S, CO, COO or Si(R¹⁴)₂ wherein R¹⁴ in each occurrence is independently a C₁₋₂₀ hydrocarbyl group. Optionally, CB contains least one sp³ hybridised carbon atom separating Ar¹ and Ar².

The linkage of an arylene repeat unit may be selected according to a desired degree of conjugation of the polymer. For example, repeat units of formula (V) or (VI) may be 2,7-linked and a repeat unit of formula (VII) may be 1,4-linked to provide conjugation across the repeat unit; other linking positions may be selected to reduce conjugation of the polymer as compared to conjugating linkages.

In some preferred embodiments, the polymer comprises a first repeat unit selected from formulae (V)-(XI), more preferably a repeat unit of formula (V) or (VI). According to these embodiments, at least one of R¹⁰, R¹¹ and R¹² is an ionic group or is an ionic substituent comprising at least one ionic group, more preferably an ionic substituent of formula (I).

In some preferred embodiments, each R¹⁰ of formula (V) or (VI) is an ionic substituent.

In some preferred embodiments, one R¹⁰ of formula (V) or (VI) is an ionic substituent and the other R¹⁰ is a non-ionic substituent.

Optionally, non-ionic substituents R¹⁰ are each independently selected from:
C₁₋₂₀ alkyl wherein one or more non-adjacent, non-terminal C atoms may be replaced with O, S, COO or CO and one or more H atoms of the alkyl may be replaced with F; and
a group of formula —(Ar¹)p wherein Ar¹ in each occurrence is independently an aryl or heteroaryl group, preferably phenyl, which is unsubstituted or substituted with one or more non-ionic substituents.

Preferably, each R¹¹ is H or both R¹¹ groups are linked to form a ring, optionally a 6 or 7 membered ring. Optionally, two R¹¹ groups are linked to form a ring in which the linked R¹¹ groups form a C₂— or C₃— alkylene chain wherein one or more non-adjacent C atoms of the alkylene chain may be replaced with O, S, NR¹³ or Si(R¹³)₂ wherein R¹³ in each occurrence is independently a C₁₋₂₀ hydrocarbyl group.

An exemplary repeat unit in which both R¹¹ groups are linked has formula (Va):

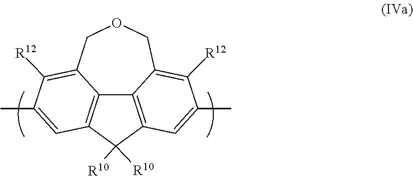

Each R¹² is preferably H or a substituent R¹⁰, more preferably H.

Optionally substituents of Ar¹, where present are selected from F, CN, NO₂ and C₁₋₂₀ alkyl wherein one or more non-adjacent, non-terminal C atoms may be replaced with O, S, COO or CO and one or more H atoms of the alkyl may be replaced with F.

In some preferred embodiments, the polymer comprises at least one repeat unit selected from formulae (V)-(XI) and at least one repeat unit selected from formulae (XII)-(XVI) wherein at least one of the repeat units of the polymer is a first repeat unit.

With reference to Formula (XI), Ar² and Ar³ are each independently unsubstituted or substituted with one or more substituents, optionally an ionic group; an ionic substituent comprising one or more ionic groups; and a non-ionic substituent, optionally a non-ionic substituent R¹⁰.

With reference to formulae (XII)-(XV), each R¹⁵, where present, is optionally selected from an ionic group; an ionic substituent comprising one or more ionic groups; and a non-ionic substituent, optionally F or a non-ionic substituent R¹⁰.

With reference to formula (XVI), R⁹, which may be the same or different in each occurrence when g is 1 or 2, is preferably selected from the group consisting of C₁₋₂₀ alkyl, Ar¹¹ and a branched or linear chain of Ar¹¹ groups wherein Ar¹¹ in each occurrence is independently substituted or unsubstituted aryl or heteroaryl.

Any two aromatic or heteroaromatic groups selected from Ar⁸, Ar⁹, and, if present, Ar¹⁰ and Ar¹¹ that are directly bound to the same N atom may be linked by a direct bond or a divalent linking atom or group. Preferred divalent linking atoms and groups include O, S; substituted N; and substituted C.

Ar⁸ and Ar¹⁰ are preferably C₆₋₂₀ aryl, more preferably phenyl that may be unsubstituted or substituted with one or more substituents.

In the case where g=0, Ar⁹ is preferably C₆₋₂₀ aryl, more preferably phenyl, that may be unsubstituted or substituted with one or more substituents.

In the case where g=1, Ar⁹ is preferably C₆₋₂₀ aryl, more preferably phenyl or a polycyclic aromatic group, for example naphthalene, perylene, anthracene or fluorene, that may be unsubstituted or substituted with one or more substituents.

R⁹ is preferably Ar¹¹ or a branched or linear chain of Ar¹¹ groups. Ar¹¹ in each occurrence is preferably phenyl that may be unsubstituted or substituted with one or more substituents.

x, y and z are preferably each 1.

Ar⁸, Ar⁹, and, if present, Ar¹⁰ and Ar¹¹ are each independently unsubstituted or substituted with one or more, optionally 1, 2, 3 or 4, substituents.

Substituents may independently be selected from an ionic group; an ionic substituent comprising one or more ionic groups; and a non-ionic substituent, optionally F or a non-ionic substituent R¹⁰.

In a preferred embodiment, g=0, and the repeat unit of formula (XVI) is a carbazole repeat unit formed by linking phenylene groups Ar⁸ and Ar⁹ by a direct bond.

Exemplary first repeat units include:

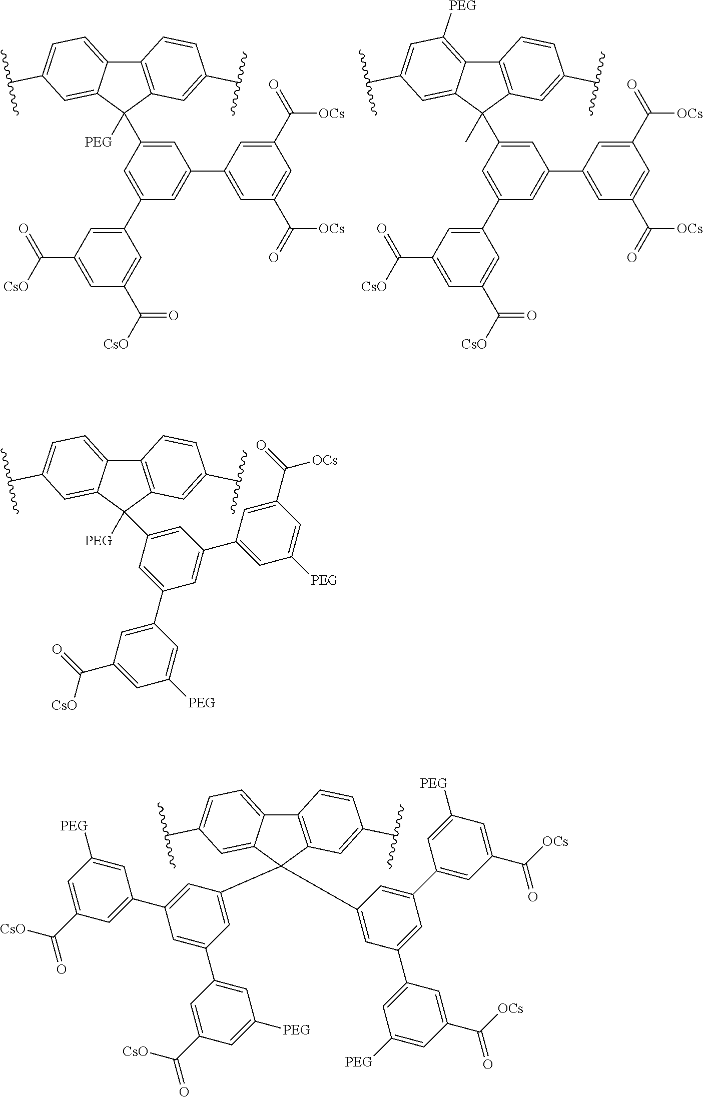

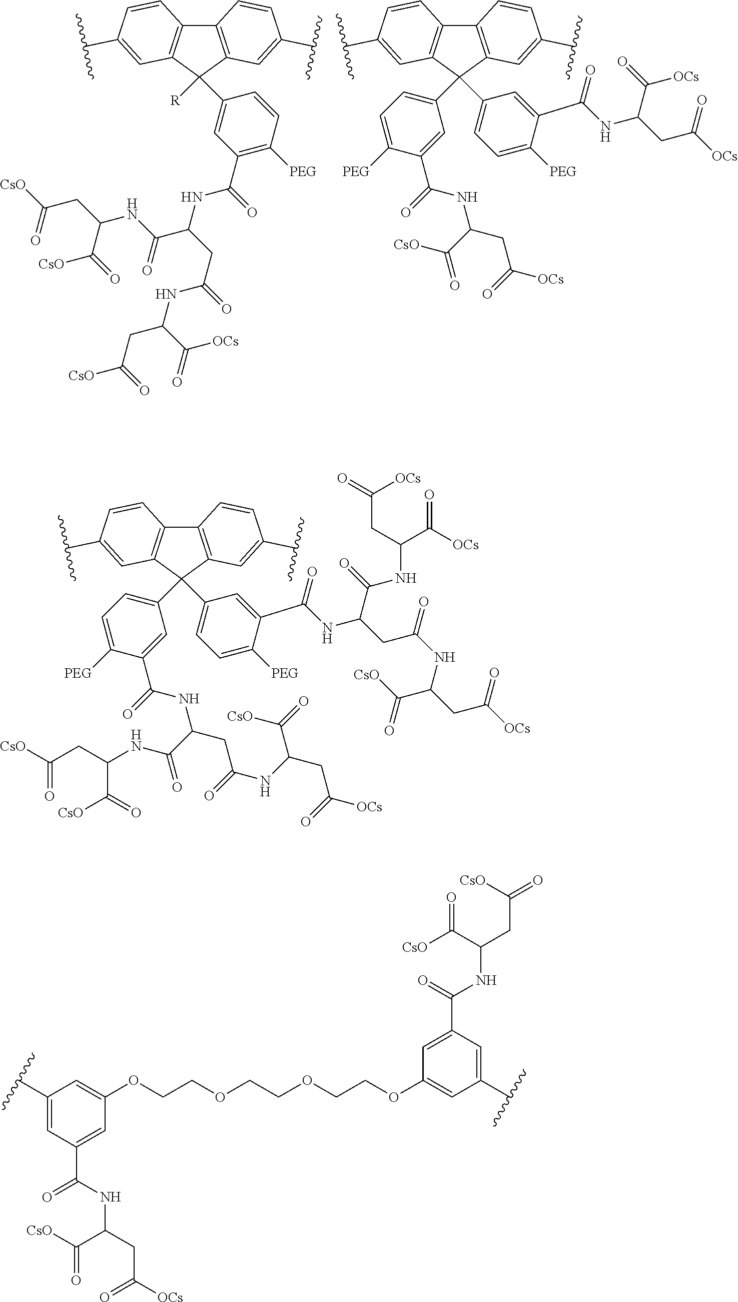

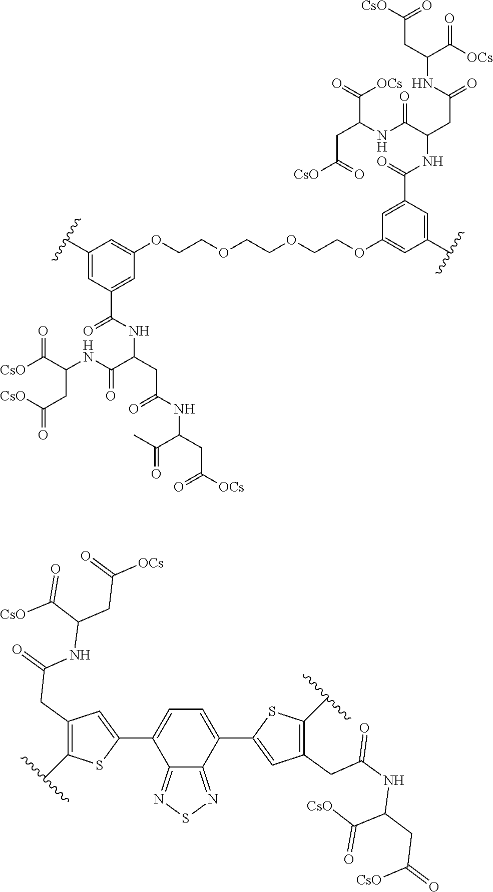

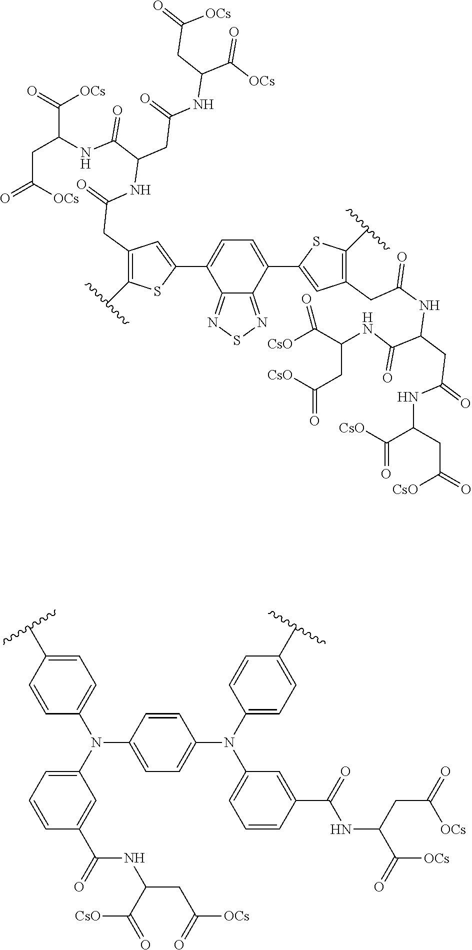

### Polymer Formation

Conjugated polymers as described herein may be formed by polymerising monomers comprising leaving groups that leave upon polymerisation of the monomers to form conjugated repeat units. Exemplary polymerization methods include, without limitation, Yamamoto polymerization as described in, for example, T. Yamamoto, “Electrically Conducting And Thermally Stable pi-Conjugated Poly(arylene)s Prepared by Organometallic Processes”, Progress in Polymer Science 1993, 17, 1153-1205, the contents of which are incorporated herein by reference and Suzuki polymerization as described in, for example, WO 00/53656, WO 2003/035796, and U.S. Pat. No. 5,777,070, the contents of which are incorporated herein by reference.

The monomers may be formed by polymerisation of monomers containing boronic acid leaving groups or esters thereof, and halide or pseudohalide (e.g. sulfonate) leaving groups. The skilled person will understand that leaving groups may be selected to control which monomers may or may not form adjacent repeat units in the polymer.

In some embodiments, ionic groups are formed after polymerisation, for example by conversion of ester groups to carboxylate groups, for example as described in WO 2012/133229, the contents of which are incorporated herein by reference. In the case of polymers containing a benzothiadiazole group, the present inventors have found that the polymer may be susceptible to some degradation upon hydrolysis, however this may be avoided by a hydrolysis reaction carried out at about 40° C., a reaction duration of 1-2 hours and 3-5 molar equivalents of base (e.g. CsOH in the case of hydrolysis to form a cesium carboxylate).

### Light-Emitting Marker

A light-emitting marker for detection of a target analyte may comprise a conjugated polymer as described herein.

In some embodiments, in use the conjugated polymer emits light upon irradiation.

In some embodiments, the conjugated polymer is used in combination with a light-emitting dye. Examples of dyes include, but are not limited to, fluorescein and fluorescein derivatives such as carboxyfluorescein, tetrachlorofluorescein, hexachlorofluorescein, carboxynapthofluorescein, fluorescein isothiocyanate, NHS-fluorescein, iodoacetamidofluorescein, fluorescein maleimide, SAMSA-fluorescein, fluorescein thiosemicarbazide, carbohydrazinomethylthioacetyl-amino fluorescein, rhodamine and rhodamine derivatives such as TRITC, TMR, lissamine rhodamine, Texas Red, rhodamine B, rhodamine 6G, rhodamine 10, NHS-rhodamine, TMR-iodoacetamide, lissamine rhodamine B sulfonyl chloride, lissamine rhodamine B sulfonyl hydrazine, Texas Red sulfonyl chloride, Texas Red hydrazide, coumarin and coumarin derivatives such as AMCA, AMCA-NHS, AMCA-sulfo-NHS, AMCA-HPDP, DCIA, AMCE-hydrazide, BODIPY and derivatives such as BODIPY FL C3-SE, BODIPY 530/550 C3, BODIPY 530/550 C3-SE, BODIPY 530/550 C3 hydrazide, BODIPY 493/503 C3 hydrazide, BODIPY FL C3 hydrazide, BODIPY FL IA, BODIPY 530/551 IA, Br-BODIPY 493/503, Cascade Blue and derivatives such as Cascade Blue acetyl azide, Cascade Blue cadaverine, Cascade Blue ethylenediamine, Cascade Blue hydrazide, CoralHue mk02, DAPI, DiA, DiD, Dil, DiO, DiR, DRAQ5, DsRED, dTomato, DyeCycle dyes, EB, ECFP, EGFP, Emerald dyes, Eosin, EYFP, Fluo-dyes, Fura dyes, FVS dyes, Hoechst33258, Indo dyes, JC-1, Kusabira-Orange, Lucifer Yellow and derivatives such as Lucifer Yellow iodoacetamide, Lucifer Yellow CH, Magnesium Green, Marina Blue, mBanana, mCherry, mOrange, mPlum, mRaspberry, mStrawberry, mTangerine, methyl Coumarin, Mitotracker Red, Na-Green, Nile Red, Oregon Green, Pacific Blue, Pacific Orange, PE dyes, PerCP dyes, Picogreen, PI, QDot dyes, R718, Rho dyes, Rhodamine Red, Riboflavin, SNARF dyes, SYBR Green, SYTOX dyes, Texas Red, TO-Pro dyes, TOTO dyes, V450, V500, Via-probe dyes, YO-Pro dyes, YOYO dyes, ZsGreen, cyanine and derivatives such as indolium based cyanine dyes, benzo-indolium based cyanine dyes, pyridium based cyanine dyes, thiozolium based cyanine dyes, quinolinium based cyanine dyes, imidazolium based cyanine dyes, Cy 3, Cy5, lanthanide chelates and derivatives such as BCPDA, TBP, TMT, BHHCT, BCOT, Europium chelates, Terbium chelates, Alexa Fluor dyes, DyLight dyes, Atto dyes, LightCycler Red dyes, CAL Flour dyes, JOE and derivatives thereof, Oregon Green dyes, WellRED dyes, IRD dyes, phycocrythrin and phycobilin dyes, Malacite green, stilbene, DEG dyes, NR dyes, CF dyes, near-infrared dyes and others known in the art.

The dye may be bound, e.g. covalently bound, to the conjugated polymer.

In some embodiments, a binding group having affinity for a target analyte is bound, preferably covalently bound, to the conjugated polymer. The binding group may be provided as a side group of a repeat unit of the polymer or as an end-group of the polymer. In some embodiments, the conjugated polymer in use, e.g. in flow cytometry, may be dissolved or dispersed in a sample to be analysed. In the case where it is dissolved, the conjugated polymer is preferably dissolved in water.

In some embodiments, the light-emitting marker is a particulate marker. In use, e.g. during flow cytometry, the particulate light-emitting marker may be dispersed in a sample to be analysed.

In some embodiments, the light-emitting marker particles comprise the conjugated polymer in collapsed form.

In preferred embodiments, the light-emitting marker particles comprise a matrix material and the conjugated polymer. The matrix material is preferably an inorganic matrix material, e.g. silica. According to these embodiments, the binding group may be bound, preferably covalently bound, to the matrix. The light-emitting particle may comprise the conjugated polymer and a light-emitting dye.

Matrix materials include, without limitation, inorganic matrix materials, optionally inorganic oxides, optionally silica. The matrix may at least partially isolate the light-emitting material from the surrounding environment. This may limit any effect that the external environment may have on the lifetime of the light-emitting material.

Light-emitting marker particles may comprise a core and, optionally, one or more shells surrounding the core.

Polymer chains of the conjugated light-emitting polymer may extend across some or all of the thickness of the core and/or shell. Polymer chains may be contained within the core and/or shell or may protrude through the surface of the core and/or shell.

The conjugated polymer may be mixed with the matrix material.

The conjugated polymer may be bound, e.g. covalently bound, to the matrix material.

In some embodiments, the particle core may be formed by polymerisation of a silica monomer in the presence of the conjugated polymer, for example as described in WO 2018/060722, the contents of which are incorporated herein by reference.

In some embodiments, the particle core comprises a core which comprises or consists of the conjugated polymer and at least one shell surrounding the inner core. The at least one shell may be silica.

Optionally, at least 0.1 wt % of total weight of the particle core consists of the conjugated polymer. Preferably at least 1, 10, 25 wt % of the total weight of the particle core consists of the conjugated polymer.

Optionally at least 50 wt % of the total weight of the particle core consists of the matrix material. Preferably at least 60, 70, 80, 90, 95, 98, 99, 99.5, 99.9 wt % of the total weight of the particle core consists of the matrix material.

The particle core as described herein is the light-emitting particle without any surface groups, e.g. binding groups or solubilising groups, thereon.

In one embodiment of the present disclosure, at least 70 wt % of the total weight of the particle core consists of the conjugated polymer and silica. Preferably at least 80, 90, 95, 98, 99, 99.5, 99.9 wt % of the total weight of the particle core consists of the conjugated polymer and silica. More preferably the particle core consists essentially of the conjugated polymer and silica.

Preferably, the particles have a number average diameter of no more than 5000 nm, more preferably no more than 2500 nm, 1000 nm, 900 nm, 800 nm, 700 nm, 600 nm, 500 nm or 400 nm as measured by dynamic light scattering (DLS) using a Malvern Zetasizer Nano ZS. Preferably the particles have a number average diameter of between 5-5000 nm, optionally 10-1000 nm, preferably between 10-500 nm, most preferably between 10-100 nm as measured by a Malvern Zetasizer Nano ZS.

Surface groups may be bound to a surface of the light-emitting particles. Exemplary surface groups include, without limitation, ether-containing groups, e.g. groups containing poly(ethyleneglycol) (PEG) chains and groups containing a binding group comprising a biomolecule.

Light-emitting particles as described herein may be provided as a colloidal suspension comprising the particles suspended in a liquid. Preferably, the liquid is selected from water, C₁₋₁₀ alcohols and mixtures thereof. Preferably, the particles form a uniform (non-aggregated) colloid in the liquid. In some embodiments, each of the first, second and any further light-emitting markers are light-emitting particles dispersed in the liquid. In some embodiments, one or more of the light-emitting markers is in particle form dispersed in the liquid and one or more of the light-emitting markers is dissolved in the liquid.

The liquid may be a solution comprising salts dissolved therein, optionally a buffer solution.

In some embodiments, the particles may be stored in a powder form, optionally in a lyophilised or frozen form.

### Functional Groups

The binding group of the light-emitting marker for binding to a target analyte may be attached to the light-emitting marker by attachment to a functional group of a precursor of the light-emitting marker.

In some embodiments, the functional group is covalently bound to the conjugated polymer.

In some embodiments, the functional group is covalently bound to a matrix material of a particulate marker precursor comprising the matrix material and the conjugated polymer.

Optionally the functional group is selected from:
amine groups, optionally —NR⁹₂ wherein R⁹ in each occurrence is independently H or a substituent, preferably H or a C₁₋₅ alkyl, more preferably H;
carboxylic acid or a derivative thereof, for example an anhydride, acid chloride or ester, acid chloride, acid anhydride or amide group;
—OH; —SH; an alkene; an alkyne; and an azide; and
biotin or a biotin-protein conjugate.

The functional group may be reacted with a biomolecule to form a linking group linking the biomolecule to the rest of the light-emitting marker, the linking group being selected from esters, amides, urea, thiourea, Schiff bases, a primary amine (C—N) bond, a maleimide-thiol adduct or a triazole formed by the cycloaddition of an azide and an alkyne.

Exemplary binding group biomolecules for binding to a target analyte include, without limitation, DNA, RNA, peptides, carbohydrates, antibodies, antigens, enzymes, proteins, hormones and combinations thereof.

In the case where the functional group is biotin, it may be conjugated to a protein, e.g. avidin, streptavidin, neutravidin and recombinant variants thereof, and a biotinylated biomolecule may be conjugated to the protein to form the light-emitting marker.

The biotinylated biomolecule may comprise an antigen binding fragment, e.g. an antibody, which may be selected according to a target antigen.

In the case of a light-emitting particle, the functional group may be bound to a surface of the light-emitting particle, e.g. bound to a matrix material of the light-emitting particle. Each functional group may be directly bound to the surface of a light-emitting particle or may be spaced apart therefrom by one or more surface binding groups. The surface binding group may comprise polar groups. Optionally, the surface binding group comprises a polyether chain. By “polyether chain” as used herein is meant a chain having two or more ether oxygen atoms.

The surface of a light-emitting particle core may be reacted to form a group at the surface capable of attaching to a functional group. Optionally, a silica-containing particle is reacted with a siloxane.

### Applications

Light-emitting markers as described herein may be used as luminescent probes for detecting or labelling a biomolecule or a cell. In some embodiments, the particles may be used as a luminescent probe in an immunoassay such as a lateral flow or solid state immunoassay. Optionally the particles are for use in fluorescence microscopy, flow cytometry, next generation sequencing, in-vivo imaging, or any other application where a light-emitting marker is brought into contact with a sample to be analysed. The analysis may be performed using time-resolved spectroscopy. The applications can medical, veterinary, agricultural or environmental applications whether involving patients (where applicable) or for research purposes.

In use the binding group of the light-emitting markers may bind to target biomolecules which include without limitation DNA, RNA, peptides, carbohydrates, antibodies, antigens, enzymes, proteins and hormones. The target biomolecule may or may not be a biomolecule, e.g. a protein, at a surface of a cell.

A sample to be analysed may brought into contact with the light-emitting marker, for example the light-emitting marker dissolved in a solution or a particulate light-emitting marker in a colloidal suspension.

In some embodiments, the sample is analysed by flow cytometry. In flow cytometry, the light-emitting marker or markers are irradiated by at least one wavelength of light, optionally two or more different wavelengths, e.g. one or more wavelengths including at least one of about 355, 405, 488, 530, 561 and 640 nm, each of which may be ±10 nm. Light emitted by the light-emitting marker(s) may be collected by one or more detectors. To provide a background signal for calculation of a staining index, measurement may be made of a light-emitting marker mixed with cells which do not bind to the light-emitting marker.

A light-emitting marker comprising or consisting of a conjugated polymer as described herein may be used as a in a method for analysing and/or sequencing nucleic acids in which the light-emitting marker is bound, suitably covalently bound, to a nucleotide.

In some embodiments of this method, a primed template nucleic acid molecule is contacted a polymerase and a test nucleotide. The test nucleotide is incorporated into the primed strand of the primed template only if it comprises a base complementary to the next base of the template strand. Emission of light from the conjugated polymer bound to the nucleotide is indicative of incorporation of the test nucleotide into the primed strand.

In some embodiments, the conjugated polymer is bound to the test nucleotide before it is brought into contact with the polymerase and the primed template nucleic acid molecule. In some embodiments, the light-emitting polymer binds to the test nucleotide after it has been incorporated into the primed strand.

The conjugated polymer may be substituted with a binding group which binds to the test nucleotide. The test nucleotide may be substituted with a complementary group for binding to the binding group. For example, one of the test nucleotide and the conjugated polymer may be functionalised with biotin and the other of the test nucleotide and the conjugated polymer may be functionalised with avidin, streptavidin, neutravidin or recombinant variants thereof.

In some embodiments, the conjugated polymer is bound to the test nucleotide by a cleavable linker, e.g. a cleavable linker formed by binding of the functional groups. Following detection of any emission from the conjugated polymer, the linker may be cleaved to separate the polymer from the primed strand and the primed strand may then be brought into contact with a further test nucleotide. Cleavage may be by treatment with a cleaving agent. Cleavage may be by irradiation. Exemplary cleavable linkers are disclosed in Leriche et al, “Cleavable linkers in chemical biology”, Bioorganic & Medicinal Chemistry, Vol. 20, Issue 2, 2012, p. 571-582, the contents of which are incorporated herein in its entirety and include, without limitation, carbamates; and groups that may be cleaved by a transition metal or a phosphine catalyst such as ethers having a disulfide alpha substituent and ethers having an azide alpha substituent.

### EXAMPLES

### Synthesis of Monomer 1

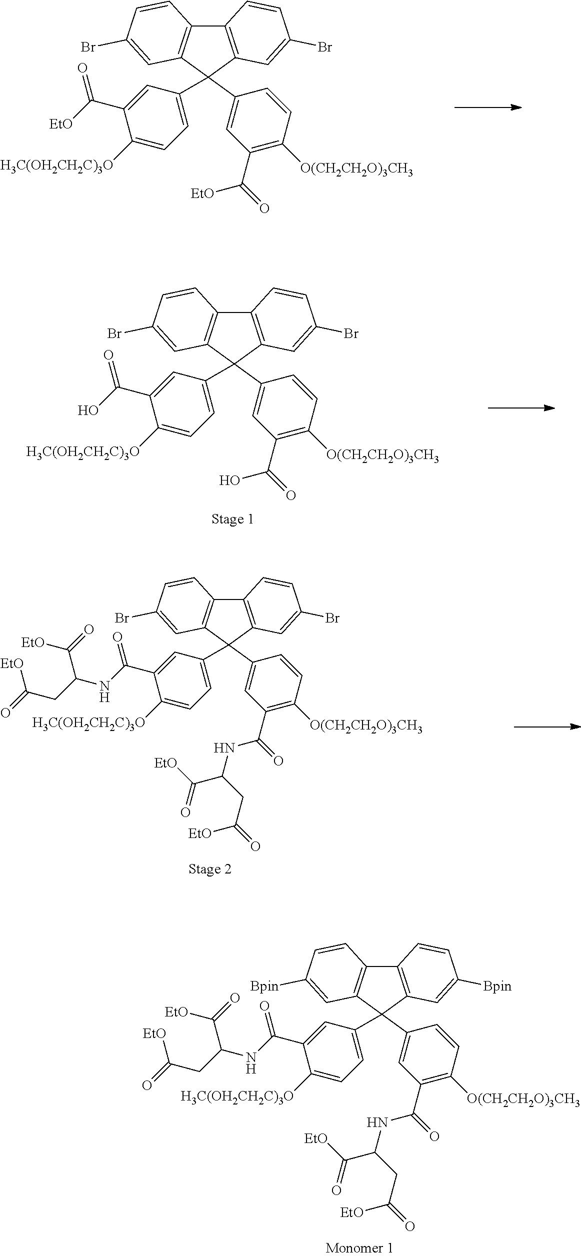

### Stage 1

The dibromide starting material is disclosed in U.S. Pat. No. 9,536,633, the contents of which are incorporated herein by reference. A flask was charged with the dibromide starting material (50 g, 52.9 mmol) and ethanol (500 mL). Sodium hydroxide (8.44 g, 211 mmol) in water (100 mL) was added and the stirred reaction mixture heated to 105° C. After TLC showed complete conversion the reaction was cooled and the solvents removed. The crude product was taken up in water and extracted with DCM. The combined aqueous layers were acidified with 3N HCl and then extracted into DCM again. The combined organics were washed with water, dried with sodium sulfate, filtered and concentrated to give stage 1 material, 44 g, 94%

### Stage 2

A flask was charged with stage 1 material (8 g, 9.0 mmol), 1,4-diethyl-2-aminobutanedioate hydrochloride (5.07 g, 22.5 mmol) and DMF (120 mL). Triethylamine (2 mL, 81.0 mmol) was added and the reaction mixture stirred for 5 min. EDC hydrochloride (6.90 g, 36.0 mmol) was added followed by anhydrous HOBt (4.86 g, 36.0 mmol) and the reaction was stirred overnight at room temperature. The reaction was diluted with water and acidified with 1.5N HCl and extracted with ethyl acetate. The organic layer was washed with water 3 times followed by brine, dried with sodium sulfate, filtered and concentrated to give crude stage 2 material. The crude product was purified by column chromatography on silica eluting with a mixture of ethanol, ethyl acetate and DCM. The product-containing fractions were triturated with ethanol and dried to give stage 2 material, 4 g, 36%.

### Monomer 1

A flash was charged with stage 2 material (8.5 g, 6.9 mmol) and toluene (250 mL). Bis(pinacolato)diboron (4.54 g, 17.9 mmol) and potassium acetate (3.7 g, 37.9 mmol) were added and the reaction mixture degassed with nitrogen for 1 h. Pd(dppf)Cl₂ (169 mg, 0.21 mmol) was added and degassing continued for a further 10 mins. The reaction was then stirred at 100° C. for 16 hours. After cooling, the reaction mixture was passed through a plug of celite and florisil and eluted with toluene and ethyl acetate. The filtrate was concentrated to give the crude product which was triturated in hexanes to give a solid. The product could be further purified by recrystallisation from toluene-ethyl acetate and hexanes to give Monomer 1 as a white powder, 6.5 g, 71%.

### Synthesis of Monomer 2

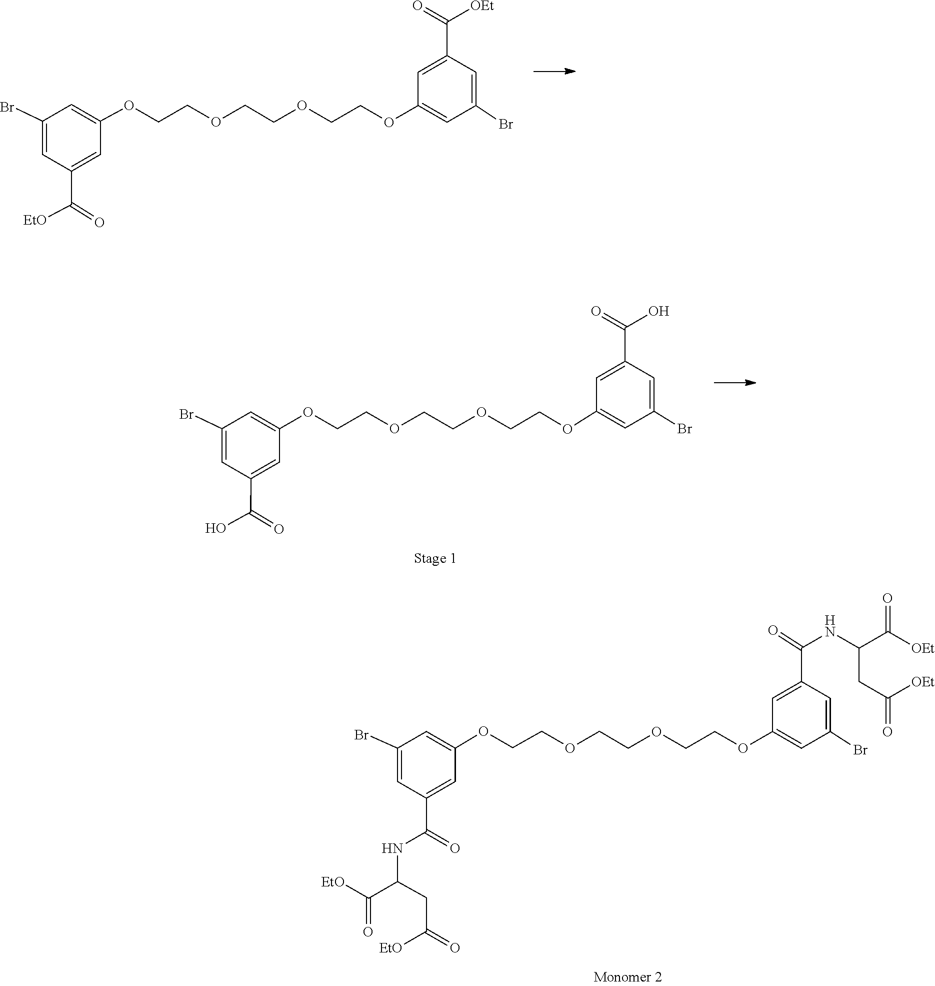 [SMILES: CCOC(=O)c1cc(Br)cc(OCCOCCOCCOc2cc(Br)cc(C(=O)OCC)c2)c1 ; O=C(O)c1cc(Br)cc(OCCOCCOCCOc2cc(Br)cc(C(=O)O)c2)c1 ; CCOC(=O)CC(NC(=O)c1cc(Br)cc(OCCOCCOCCOc2cc(Br)cc(C(=O)NC(CC(=O)OCC)C(=O)OCC)c2)c1)C(=O)OCC]

### Stage 1

The dibromide starting material is disclosed in WO 2021/001663, the contents of which are incorporated herein by reference. A flask was charged with the dibromide starting material (35 g, 57.9 mmol) and ethanol (400 mL). Sodium hydroxide (9.24 g, 231 mmol) in water (100 mL) was added and the stirred reaction mixture heated to 105° C. for 6 h. The reaction was cooled and the solvents removed. The crude product was taken up in water and extracted with DCM. The combined aqueous layers were acidified with 3N HCl and stirred until a solid precipitated. The solid was filtered and washed with water and ethanol to give stage 1 material, 23g, 72%.

### Monomer 2

A flask was charged with stage 1 material (15 g, 27.3 mmol), 1,4-diethyl-2-aminobutanedioate hydrochloride (15.3 g, 68.2 mmol) and DMF (225 mL). DIPEA (42.7 mL, 245 mmol) was added and the reaction mixture stirred for 5 min. EDC hydrochloride (15.7 g, 81.9 mmol) was added followed by anhydrous HOBt (11.0 g, 81.9 mmol) and the reaction was stirred overnight at room temperature. The reaction was poured into water and stirred for 0.5 h to produce a gummy solid. The water was decanted and the solid dissolved in ethyl acetate and washed with water, dilute HCl and brine, dried with sodium sulfate, filtered and concentrated to give crude monomer 2. The crude product was purified by column chromatography on silica eluting with a mixture of ethanol, ethyl acetate and DCM. The product-containing fractions were recrystallised from toluene-acetonitrile to give Monomer 2, 11 g, 45%.

### Polymer Formation

Polymers were made by polymerisation of monomers set out in Table 1 by a Suzuki polymerisation process as set out in U.S. Pat. No. 9,536,633.

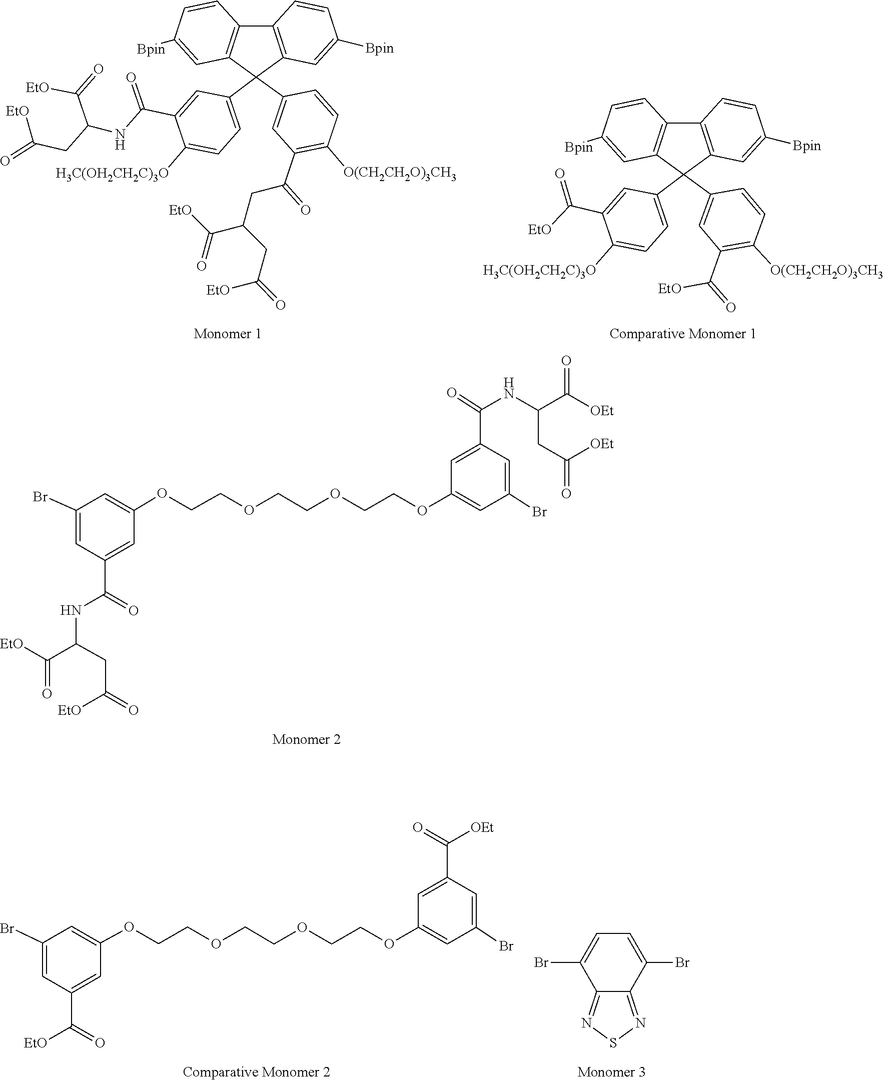

TABLE 1

| | Mono- | Comparative | Mono- | Comparative | Mono- |
|---|---|---|---|---|---|
| | mer 1 | Monomer 1 | mer 2 | Monomer 2 | mer 3 |
| Polymer 1 | 50% | n/a | 10% | n/a | 40% |
| Comparative | n/a | 50% | n/a | 10% | 40% |
| polymer 1 | | | | | |

The polymers were then hydrolysed with cesium hydroxide to convert the ethyl ester groups to cesium carboxylate groups as described in U.S. Pat. No. 9,536,633.

The solubility of the hydrolysed polymers in water and the filterability of the solutions through 0.45 μm syringe filters were compared. Results are set out in Table 2.

TABLE 2

| | 10 mg/mL | | 4 mg/mL | | 2 mg/mL | | 1 mg/mL | |
|---|---|---|---|---|---|---|---|---|
| | Soluble | Filters | Soluble | Filters | Soluble | Filters | Soluble | Filters |
| Polymer 1 | Y - | Y | n/a | n/a | n/a | n/a | n/a | n/a |
| | slightly | | | | | | | |
| | viscous | | | | | | | |
| Comparative | N | n/a | N - | N | Y - very | N | Y - | Y |
| polymer 1 | | | forms | | viscous | | viscous | |
| | | | gel | | | | | |

As can be seen from Table 2, Polymer 1 was readily soluble in water at 10 mg/mL giving a solution that could be filtered through a 0.45 μm syringe filter whereas Comparative Polymer 1 was only partially soluble in water at 10 mg/mL. Dilution to 4 mg/mL gave a gel after being placed on a hotplate for ˜1 hour. Comparative Polymer 1 required dilution to 1 mg/ml to give a water solution that could be filtered through a 0.45 μm syringe filter.

1. A light-emitting particle comprising a silica matrix material and a conjugated polymer comprising a first repeat unit substituted with at least three ionic groups.

2. The light-emitting particle according to claim 1 wherein each ionic group is an anionic group.

3. The light-emitting particle according to claim 1 wherein the polymer is a copolymer comprising the first repeat unit and at least one further repeat unit.

4. The light-emitting particle according to claim 1 wherein the first repeat unit is substituted with at least one ionic substituent carrying at least 2 ionic groups.

5. The light-emitting particle according to claim 4 wherein the at least one ionic substituent is substituted with at least three ionic groups.

6. The light-emitting particle according to claim 4 wherein the first repeat unit substituted with at least two of the ionic substituents.

7. The light-emitting particle according to claim 1 wherein the ionic substituent has formula (I):
-L-(R¹)x (I)
wherein each R¹ is independently an ionic group; x is at least 1; and L is a linking group linking the ionic group or ionic groups to R¹ to the first repeat unit.

8. The light-emitting particle according to claim 1 wherein the first repeat unit is selected from repeat units of formulae (V)-(XVI): 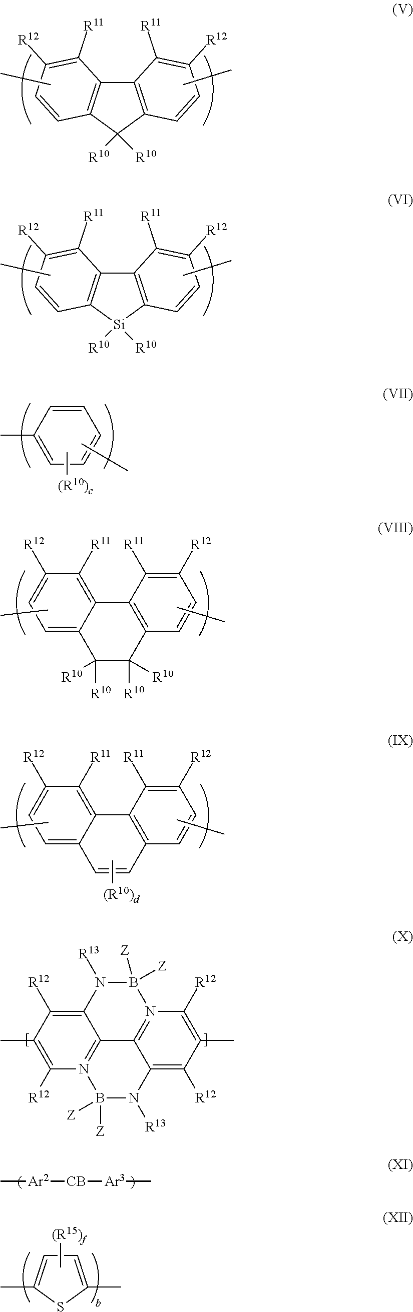 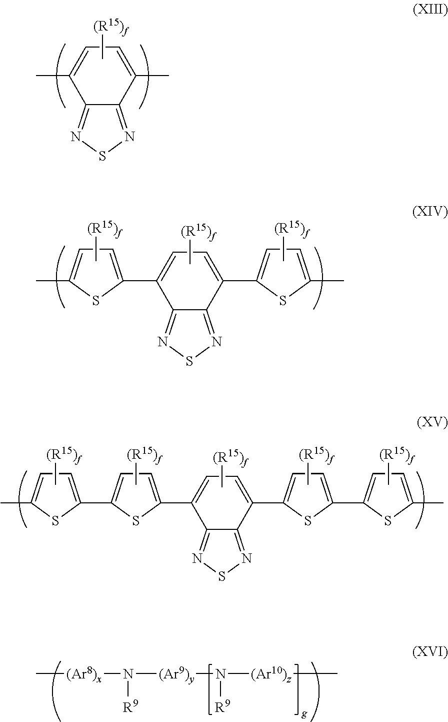
wherein:
R¹⁰ in each occurrence is independently a substituent;
R¹¹ in each occurrence is independently H or a substituent and two R¹¹ groups may be linked to form a ring;
R¹² independently in each occurrence is H or a substituent;
R¹³ independently in each occurrence is a C₁₋₂₀ hydrocarbyl group;
R¹⁵ independently in each occurrence is a substituent;
Z in each occurrence is independently a substituent.
c is 0, 1, 2, 3 or 4;
d is 0, 1 or 2;
f independently in each occurrence is 0, 1 or 2;
Ar⁸, Ar⁹ and Ar¹⁰ in each occurrence are independently selected from substituted or unsubstituted arylene or heteroarylene;
g is 0, 1 or 2;
R⁹ independently in each occurrence is a substituent,
x, y and z are each independently 1, 2 or 3;
Ar² and Ar³ each independently represent a C₆₋₂₀ arylene group or a 5-20 membered heteroarylene group which is unsubstituted or substituted with one or more substituents; and
CB represents a conjugation-breaking group which does not provide a conjugation path between Ar² and Ar³.

9-11. (canceled)

12. A method of forming the light-emitting particle according to claim 1 wherein a material for forming the silica is reacted in the presence of the conjugated polymer dissolved in an alcoholic solvent.

13. A light-emitting marker comprising the light-emitting particle according to claim 1 and a binding group comprising a biomolecule.

14. A precursor particle of the light-emitting marker according to claim 13 comprising a functional group covalently bound to the matrix material and capable of binding to the biomolecule.

15. The precursor particle according to claim 14 wherein the functional group comprises biotin.

16. A method of forming the light-emitting marker according to claim 13 comprising binding the biomolecule to a functional group of a precursor particle comprising the functional group covalently bound to the matrix material and capable of binding to the biomolecule.

17. A formulation comprising light-emitting particles according to claim 1 or precursor particles thereof dissolved or dispersed in one or more solvents.

18. A method of identifying a target analyte in a sample, the method comprising irradiating the sample to which has been added a light-emitting marker according claim 13 configured to bind to the target analyte; and detecting emission from the light-emitting marker,

19. The method according to claim 18 wherein the method is a flow cytometry method and the target analyte is a target cell.

20. A method of sequencing nucleic acids comprising contacting a primed template nucleic acid molecule with a polymerase and a test nucleotide;
incorporating the test nucleotide into a primed strand of the primed template only if it comprises a base complementary to the next base of the template strand;
irradiating the primed strand; and
determining from luminance of the primed strand if the test nucleotide has been incorporated into the primed strand,
wherein the test nucleotide of the irradiated primed strand is bound to a light-emitting marker according to claim 13.

21. The light-emitting particle according to claim 1 wherein the at least 3 ionic groups are cationic groups comprising a counterion M⁺ wherein M⁺ is a metal cation or an organic cation.
